(19) United States
(12) Patent Application Publication
(10) Pub. No.: US 2018/0340947 A1
(43) Pub. Date: Nov. 29, 2018

(54) INVOLVEMENT OF THE BUFODIENOLIDES IN THE DIAGNOSIS AND TREATMENT OF POST-TRAUMATIC STRESS DISORDER (PTSD)

(71) Applicant: Jules B. Puschett, Temple, TX (US)

(72) Inventor: Jules B. Puschett, Temple, TX (US)

(21) Appl. No.: 15/987,039

(22) Filed: May 23, 2018

Related U.S. Application Data
(60) Provisional application No. 62/509,836, filed on May 23, 2017.

(51) Int. Cl. G01N 33/92; A61K 31/585

(52) U.S. Cl. CPC G01N 33/92; A61K 31/585; G01N 2800/52; G01N 2800/30; G01N 2405/00

(57) ABSTRACT

The method of this invention relates a method of diagnosing a patient as having post-traumatic stress disorder (PTSD) and/or treating the patient therapeutically for PTSD. In one embodiment, the treatment involves administering a therapeutically effective amount of bufodienolide antagonist to the patient.

### PRIORITY CLAIM

This application claims priority to U.S. Application No. 62/509,836 filed May 23, 2017 which is incorporated herein by reference in its entirety.

### BACKGROUND

The risk for the individual's exposure to life-threatening events like natural disasters, accidents, attacks, assaults and other acts of violence, not to mention terrorist attacks, has considerably increased during the past decades. All these incidents can trigger the development of stress-related disorders. This includes the so called posttraumatic-stress disorder (PTSD), which is characterized by persistent and frightening re-experiencing of the traumatic event, accompanied by severely impairing sleep disturbances and avoidance behavior. Besides PTSD, which is directly linked to a specific traumatic experience, also anxiety disorders and depression can be triggered by adverse events.

Posttraumatic stress disorder (PTSD) occurs in only a small proportion of those exposed to traumatic events. Several risk factors have been implicated in the development of PTSD. These include age at traumatization, gender, earlier childhood exposures (“pre-traumatic”) to adversity, personality characteristics, and familial psychopathology, including PTSD (Yehuda and LeDoux, Neuron 2007, 56:19-32). There is evidence from twin studies that genetic factors contribute to the risk for PTSD (Stein et al., Am J Psychiatry 2002, 159:1675-81; True et al., Arch Gen Psychiatry 1993, 50:257-64; Xian et al., Drug Alcohol Depend 2000, 61:95-102).

There remains a need for additional diagnostic and prognostic methods for PTSD, as well as additional methods for treating, ameliorating, or preventing PTSD.

### SUMMARY

The method of this invention relates a method of diagnosing a patient as having post-traumatic stress disorder (PTSD) and/or treating the patient therapeutically for PTSD. In one embodiment, the treatment involves administering a therapeutically effective amount of bufodienolide antagonist to the patient.

The diagnostic portion of the present invention may involve measuring marinobufagenin (MBG) in a patient sample. The patient sample can be urine, blood, blood serum or blood plasma, or cerebrospinal fluid.

The preferred method involves determining if a patient has a propensity for PTSD by obtaining a sample from the patient, determining the concentration of marinobufagenin in the sample, and comparing the concentration of marinobufagenin with the marinobufagenin concentration in a similar sample in normal patients or a reference level. If the marinobufagenin concentration is substantially above the concentration of a normal patient or reference, this indicates that a propensity for PTSD exists and therapeutic action can be initiated. Assays for determining a level of MBG or if a propensity for PTSD exists is represented by use of an immuno-fluorescent ELISA assay, for example, to provide or determine the amount of marinobufagenin in the patient sample, e.g., urine, blood or cerebrospinal fluid specimen. In a preferred embodiment of the present invention, it is determined that a propensity for PTSD exists if the elevation of marinobufagenin is at least about 30 percent over that of a normal patient or reference.

The diagnostic tests and therapeutic treatment may be repeated periodically to determine trends. If the marinobufagenin concentration continues to increase, this reinforces the conclusion that a propensity for PTSD exist. If it decreases, comparison of the concentration with normal patients will facilitate a determination of reduced concern.

As employed herein, the term “normal patient(s)” means a group of non-traumatized subjects matched for age and sex.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A. Endogenous cardiac glycosides cardenolides (left column) bufodienolides (right column).

FIG. 1B. Chemical structures of resibufogenin (RBG) (above) and marinobufagenin (MBG) (below). The compounds differ in a hydroxyl group at the beta-5 position of the RBG molecule.

FIGS. 2A-2C. Micrographs of GFAP (glial fibrillary acidic protein) immuno-fluorescence. GFAP antibody labels the intermediate filaments of most astrocytes in the brain. In the sham animals (FIG. 2A), a normal distribution of GFAP-labeled astrocytes is seen. At 24 hours after an impact acceleration injury (IAI; FIG. 2B), a noticeable scar is observed (arrows) in layers 2 and 3 of the cortex. There is also a patchy loss of GFAP-labeled astrocytes throughout cortex and the vasculature (arrowheads) appears damaged. The loss of GFAP immunoreactivity and the appearance of astrocyte hypertrophy are indicative of a neuroinflammatory response to injury. As seen in the third image (FIG. 2C), most of these alterations are ameliorated if the animals are treated with RBG at 1 hour after IAI (IAI+RBG). A glial scar was not observed in the IAI+RBG animals. The RBG treatment also reduces the decrease in GFAP immunoreactivity and a greater proportion of GFAP-labeled astrocytes have a normal, rather than hypertrophied appearance.

FIG. 3. MBG levels in concussed civilian subjects. Initial values are provided on the left and those obtained 3 months later are on the right.

FIG. 4. Marinobufagenin (MBG) levels in concussed athletes (closed circles) compared to those obtained prior to spring training (diamonds).

FIG. 5. Preliminary studies of urinary MBG values obtained in 10 controls and 14 PTSD patients.

### DESCRIPTION

Bufodienolides were discovered in amphibians and extracted from plants. They are thought to act by virtue of their ability to inhibit Na+/K+-ATPase activity (Flier et al., Science 1980, 208:503-05). Several bufodienolides have been suggested as candidate sodium pump ligands (SPLs) in mammals, including marinobufagenin (MBG), which acts in vitro as a vasoconstrictor (Fedorova et al., Am. J. Hypertens. 1997, 10:929-35; Lopatin et al., J. Hypertens. 1999, 17:1179-87). Enhanced MBG production occurs in pathological states associated with fluid retention, including essential and salt-sensitive hypertension, preeclampsia, and uremic cardiomyopathy (Gonick et al, Clin. Exp. Hypertens. 1998, 20: 617-27, Bagrov et al., Hypertension 1998, 31:1097-1103; Fedorova et al., Hypertension 2001, 37:462-66; Lopatin et al., J. Hypertens. 1999, 17:1179-87; Fedorova et al, Circulation 2002; 105: 1122-27; Kennedy et al., Hypertension 2006, 47:448-495).

Bufodienolides (e.g., Formulas I, II, and III below) and related substances called the cardenolides (e.g., Formulas IV, V, and VI below) are termed the “cardiotonic steroids” or “cardiac glycosides.” They are similar in general structure but different in specific structure and function. However, all of the cardiotonic steroids have the ability to inhibit the ubiquitous enzyme sodium/potassium ATPase (Na+/K+ATPase).

Certain aspects described herein relate in part to marinobufagenin (MBG), the most extensively studied of the bufodienolides. MBG is elevated in disturbances that result from excessive volume expansion (Gonick et al. Clin. Exp. Hypertens. 20: 617-627, 1998; Ianosi-Irimie et al. Clin. Exp. Hypertens. 8: 605-617, 2005; Vu et al. Exp. Biol. Med. 231: 215-220, 2006). An immunoassay has been developed to measure MBG in both serum and urine (Abi-Ghanem et al. Journal of Immunoassay and Immunochemistry 32: 31-46, 2011). Certain embodiments are directed to the measurement of MBG for determining whether or not excessive volume expansion exists.

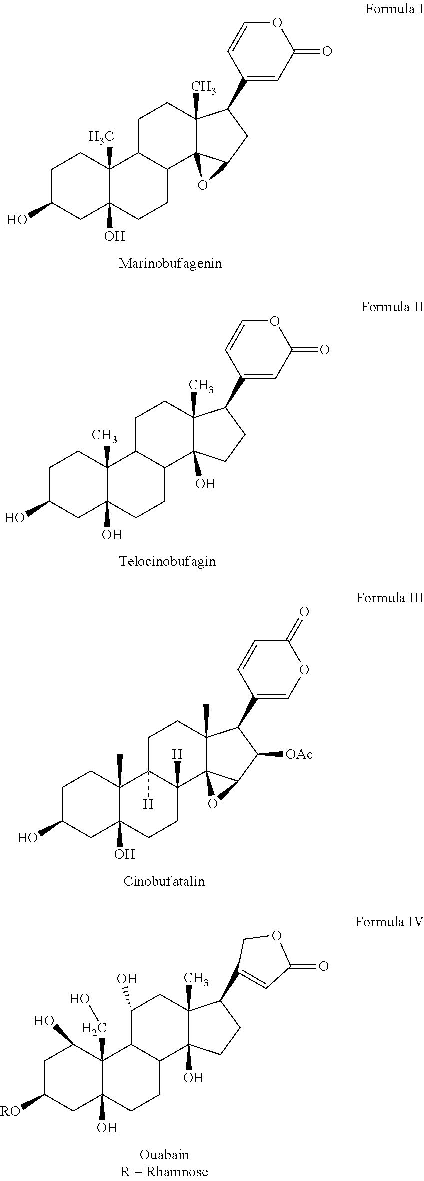

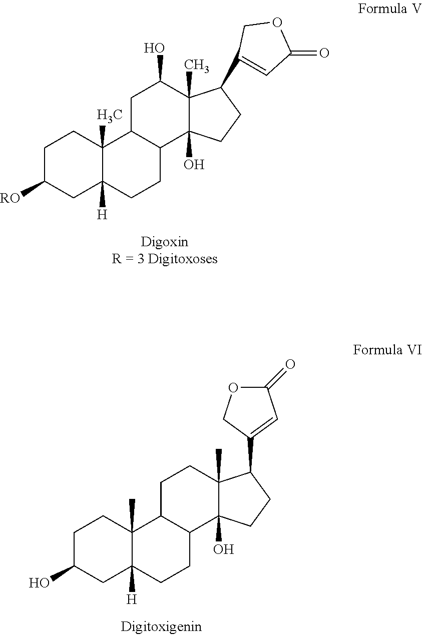

MBG is elevated in other syndromes such as preeclampsia and traumatic brain injury. It has been shown that the administration of the antagonist of MBG, resibufagenin (RBG), is therapeutically effective. RBG administration results in an amelioration of tissue injury in a rodent brain contusion model when given 1 hour after the insult and prevents preeclampsia in animal models. RBG antagonizes MBG activity and may be an effective treatment for hypertension, ARDS, traumatic brain injury, and preeclampsia. Certain embodiments are directed to treating a patient having elevated levels of MBG by administering an MBG antagonist or anti-MBG agent. In certain aspects the anti-MBG agent is resibufagenin (RBG).

### I. Post-Traumatic Stress Disorder (PTSD)

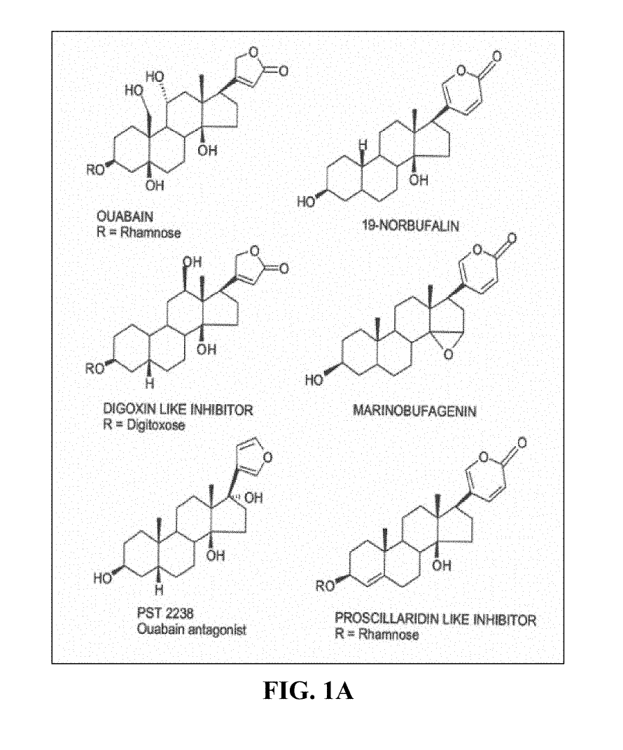

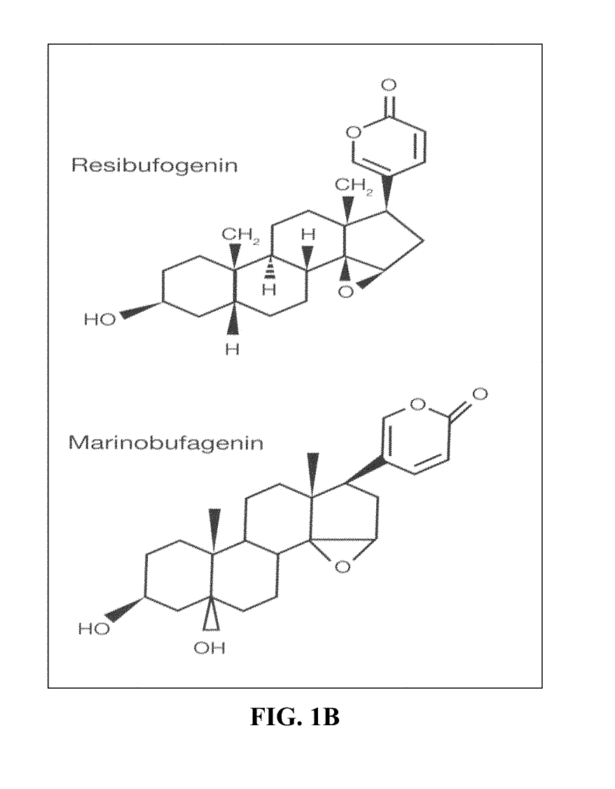

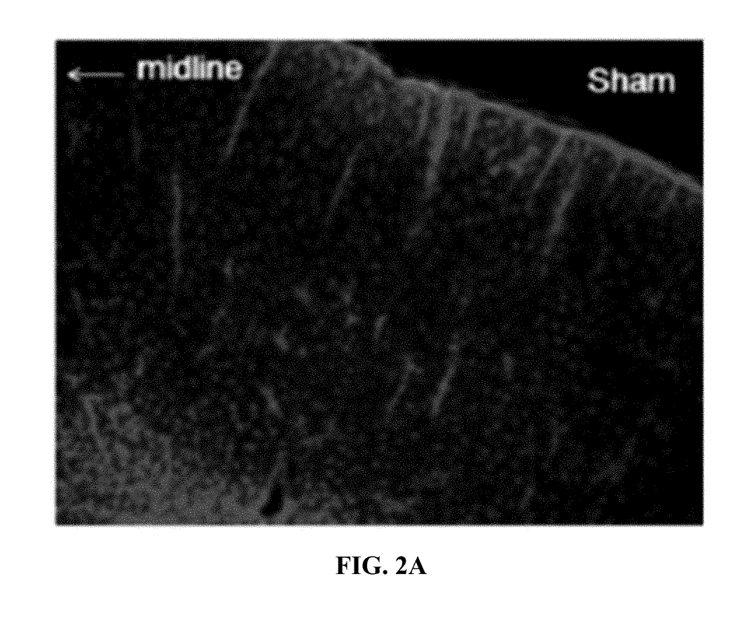

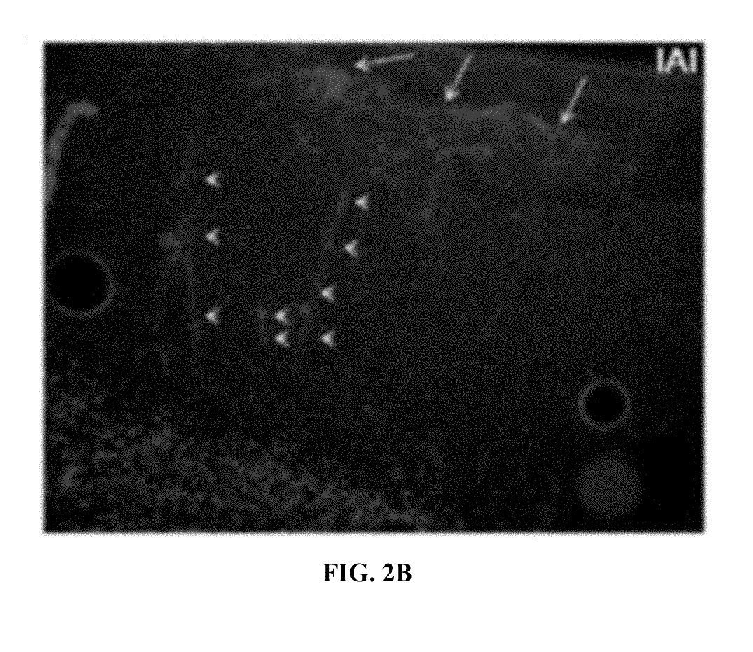

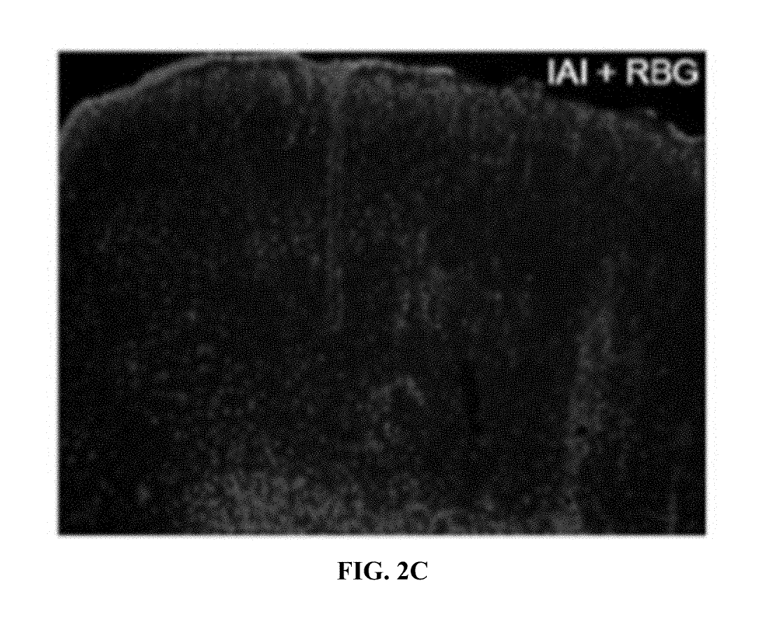

There are estimated to be between 300,000 to 350,000 veterans of the Middle East conflicts who currently suffer from PTSD related to wartime service. However, there are no proven methods to diagnose and stage the results of both physical and psychological trauma leading to PTSD, nor are there successful preventative or treatment methods. The interest in these military-associated problems has been accelerated by their potential application also to concussion in civilian life, especially to amateur, collegiate, and professional sports. Thus far, no solution has been enunciated nor has been proven (Meier and Ivory, Jul. 3, 2015, The New York Times). Furthermore, although initial indications regarding any possible connections between traumatic brain injury (TBI) and post-traumatic stress disorder (PTSD) suggested separate clinical processes (Sbordone and Liter, Brain Injury 1995, 9:405-12; Hickling et al., Brain Injury 1998, 12:265-74), recent substantial evidence indicates that TBI may lead to the development of PTSD (Boyle et al., Archives of Physical Medicine and Rehabilitation. 2014, 95(3 Suppt 2):S230-37; Spielberg et al., Biological Psychiatry 2015, 78:210-16; Always et al., Journal of Neurotrauma (doi:10.1089/neu.2015, 3992); Ruff et al., BMJ Open 2012, 2:e000312; Bryant et al., British Journal of Psychiatry doi:10.1192/bjp.bp. 114. 145516; Hoge et al., N Engl J Med 2008, 358:453-63; Gil, Sharon et al., Psychological Trauma: Theory, Research, Practice, and Policy 8.1, 2016:49-54; Bryant and Marossze, Brain Injury 14.2 2000:175-180; Ragsdale et al., Journal of Anxiety Disorders 2013, 27.4:420-26; Morey et al., Human Brain Mapping 2012, 34.11:2986-999; Bazarian et al., Journal of Head Trauma Rehabilitation 2013, 28.1:1-12; McNeil and Greenwood, Cognitive Neuropsychiatry 1996, 1.3:239-46; McMillan, Brain Injury 1996, 10.10:749-58; King, Journal of Head Trauma Rehabilitation 1998, 13.1:86; Bryant and Marossze, Brain Injury 2000, 14.2:175-80; Warden et al., The Journal of Neuropsychiatry and Clinical Neurosciences 1997, 9.1:18-22; Morissette et al., Rehabilitation Psychology 2011, 56.4:340-50; Williams et al., Journal of Traumatic Stress 2002, 15.5:397-400; Ruff et al., BMJ Open 2012, 2.2:e000312; Wolf et al., Journal of Traumatic Stress 2015, 28.4:339-47; Powell et al., Journal of Traumatic Stress 2015, 28.4:322-29; Alway et al. Journal of Neurotrauma 2016, 33.9:825-31; Schneiderman et al., American Journal of Epidemiology 2008, 167.12:1446-52). It has been determined that a circulating cardiac glycoside (or “cardiotonic steroid”) called marinobufagenin (MBG) (FIG. 1), acts as both an indicator and initiator of the inflammatory process involved in TBI (FIG. 1) (Shapiro et al., The American Society for Clinical Pharmacology and Therapeutics, Dallas, Tex., Mar. 2-5, 2011. ASCPT Annual Meeting Program Booklet, p. 78; Puschett et al., Medical Research Archives 2015, ISSN 2375-1924; Oliver et al., J Experimental Neuroscience 2015, 9:67-72). This substance, MBG, is a member of a family of compounds called the bufodienolides (FIGS. 1A and 1B). MBG has been found to be elevated in both the blood and urine of experimental animals and patients who have been subjected to concussion. Furthermore, MBG has been determined to cause vascular leak (Uddin et al., Am J Nephrol 2009, 30:26-33; Uddin et al., Am J Physiol Regul Integr Comp Physiol 2009, 296:R1726-R1734; Ing et al., Am J Physiol, Regulatory, Integrative and Comparative Physiology 2014, 306:R9158-R924) including disruption of the blood/brain barrier (Ing et al., Am J Physiol, Regulatory, Integrative and Comparative Physiology 2014, 306:R9158-R924). It has been determined that a compound related to MBG, resibufagenin (RBG), antagonizes the effects of MBG in an animal model of TBI (FIG. 2, Table 1). It is contemplated that RBG can be used as a preventative and/or therapeutic strategy in this disorder.

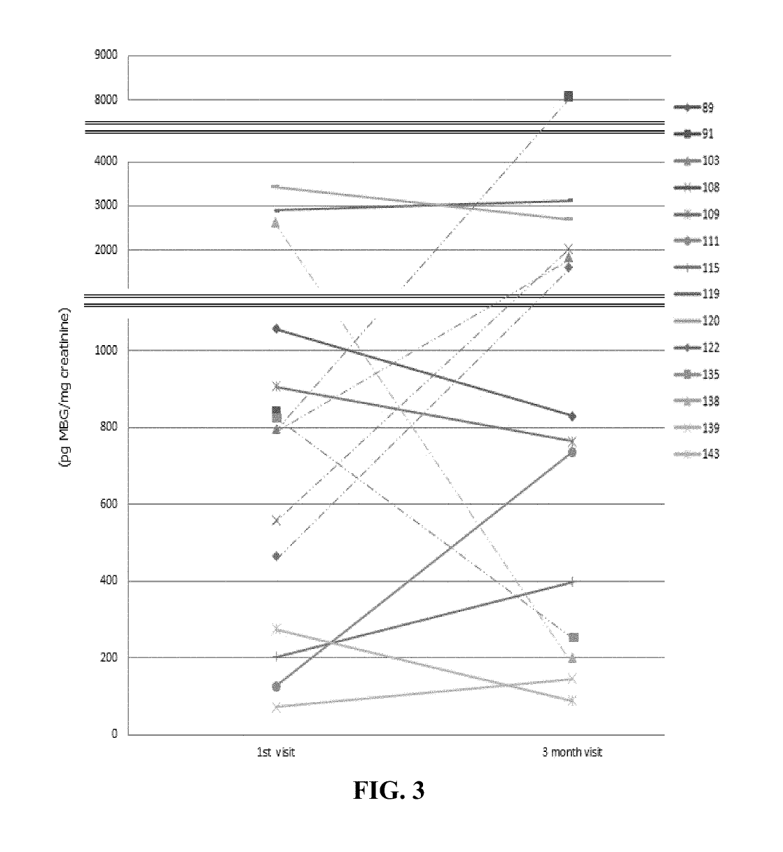

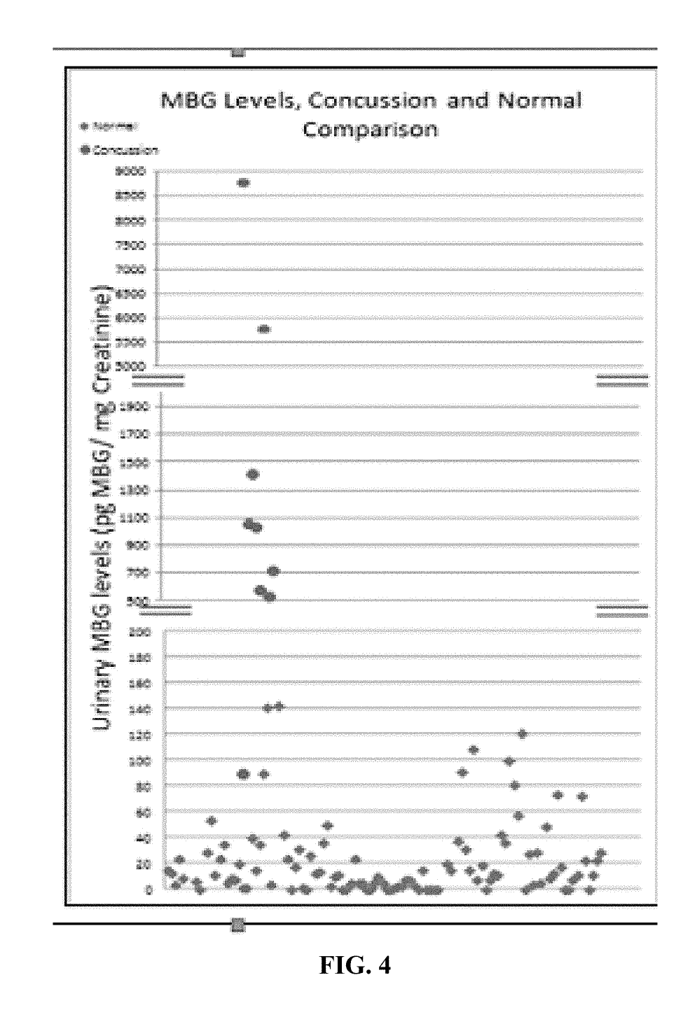

In a 3-month longitudinal study of civilians who were victims of brain trauma due to traffic accidents or altercations (FIG. 3) (McMillan, Brain Injury 1996, 749-58) MBG levels improved in only approximately ⅓ of patients over time, yet levels were unchanged in approximately another ⅓, and higher in a final ⅓ of subjects (FIG. 3). In another study MBG levels in college athletes before participation in football activities and at several time points post-concussion, MBG values increased several fold from control levels. Furthermore, MBG values increased again once players returned to the playing field (FIG. 4). These data support the view that cerebral inflammation can continue post-concussion.

There is controversy among neuroscientists as to whether or not PTSD is a complication of TBI or is a separate condition (Bryant et al., British Journal of Psychiatry doi:10.1192/bjp.bp.114.145516). However, there are now a number of studies that provide evidence for the view that PTSD represents one of the outcomes of TBI, especially multiple episodes of TBI. However, whereas the results of neurocognitive tests generally returned to normal by 7-10 days post-concussion, MBG levels were still elevated.

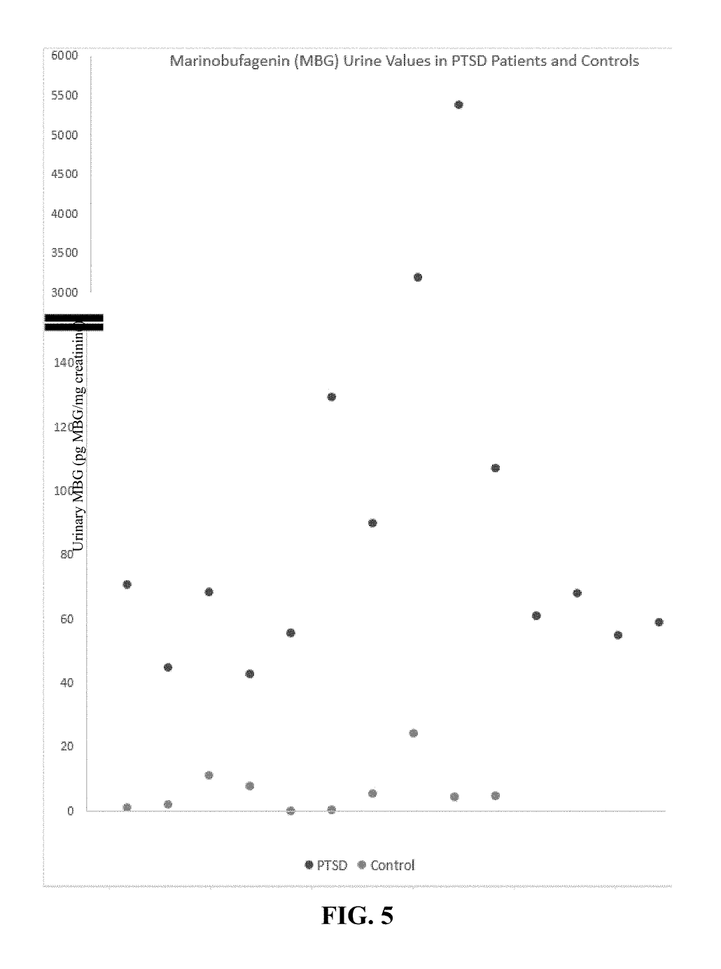

It is a premise of the current invention that persistent inflammation is the cause in most cases of PTSD. The measurement of MBG levels can assist in the determination as to whether or not persistently elevated levels herald and identify continued inflammation in the brain tissue representing an ongoing process leading eventually to PTSD. FIG. 5 presents data from studies of urine MBG levels in 10 control subjects and 14 patients with PTSD.

TABLE 1
Urinary MBG excretion in sham and
traumatic brain injured animals

| Group | Sham | TBI | TBI + RBG |
|---|---|---|---|
| N | 10 | 10 | 10 |
| Values | 592.8 ± 79.3 | 1340.9 ± 308.5 | 475.3 ± 56.1 |

P values: sham vs. TBI = 0.04; TBI vs. TBI + RBG = 0.02; sham vs. TBI + RBG = <0.05

The present invention relates to a method of identifying a predisposition for developing posttraumatic stress disorder (PTSD) in a subject comprising assessing in a sample obtained from said subject the level of MBG, wherein an increase in MBG as compared to a normal or reference level is indicative of a predisposition for developing PTSD. Further, the invention relates to a method of preventing or treating PTSD by administering an antagonist of MBG. In particular aspects the MBG antagonist is RBG.

Accordingly, the present invention relates in a first embodiment to a method of identifying a predisposition for developing posttraumatic stress disorder (PTSD) in a subject comprising assessing in a sample obtained from the subject the level of MBG, wherein an increase in the level is indicative of a predisposition for developing PTSD.

The term “predisposition for a disease” is established in the art and used herein analogously. The term “predisposition for developing posttraumatic stress disorder (PTSD)” as used in accordance with the present invention describes the status of a patient at risk to develop said disease once exposed to a trauma. As used herein, the predisposition to develop PTSD is based on MBG levels in a subject. The predisposition to develop PTSD may be diagnosed according to a method of the invention upon exposure to a traumatic event as well as prior to exposure to said event.

“Posttraumatic stress disorder” as used herein relates to a condition characterized by the development of characteristic symptoms following exposure to a traumatic stressor such as direct personal experience of an event.

The term “sample” as used herein refers to a biological sample, such as, for example, cells, tissues (from any organ including post-mortem brain tissue), or fluids (including serum, whole blood, cerebrospinal fluid, lymph, saliva, milk, pus, urine, feces), which has been isolated or obtained from an individual. In particular aspects the sample is urine or blood, including various blood fractions.

With the diagnostic or prognostic method of the invention, an imminent risk for the development of PTSD can be identified, and medical interventions can be initiated at a very early stage of the disease process. Such a diagnostic method can be applied to individuals, who, for instance, just recently experienced a traumatic event. Individuals with an increase in MBG can be administered therapeutic or preventive medical attention to avoid or ameliorate the development of PTSD. This assessment can identify subjects who will benefit from preventive medical intervention, thus saving the health of the individual and reduce costs for the health-care system. Methods for the assessment of MBG levels are well-known in the art, such as ELISA.

A further embodiment of the invention relates to a method of selecting a therapy to prevent or treat PTSD comprising the steps of: (a) identifying a predisposition for developing posttraumatic stress disorder according to the method of the invention; and (b) selecting a therapy based on the results obtained in the preceding step. Advantageously, PTSD can be predicted or diagnosed and preventive or therapeutic measures can be applied accordingly.

A variety of therapies exist to treat PTSD. Generally, physicians choose from psychotherapeutic interventions alone or in combination with psychopharmacological drugs from the type of a selective serotonin reuptake inhibitor (S SRI) or of a benzodiazepine (BZD) for severe forms of the disorder. While psychotherapeutic interventions are costly in terms of money and time, SSRIs and BZD also have impediments, especially with respect to their side-effects. SSRIs can induce a number of medical problems related to the peripheral effects of these drugs. This includes nausea, stomach upset, muscle pain, tachycardia or heart arrhythmia, diminished sexual interest and/or performance, and sleep disturbances. BZD are usually medically well tolerated, but can produce psychomotor side effects and memory disturbances, as well as and foremost can induce tolerance, withdrawal symptoms, and dependence. If an ongoing development of PTSD can be diagnosed prior to the development of full symptoms, the outcome of the disorder would be more favorable (Breslau, Clin Psychiatry 2001, 62(17): 55-9).

The step of selecting a therapy may comprise the additional step of applying and monitoring a therapy and based on its outcome select a therapy. For example, subjects that are diagnosed to be at risk for developing PTSD and put on a prophylactic therapy can be monitored and, if necessary, their therapy subsequently be adjusted or changed depending on whether PTSD symptoms occur and to which extent or not. Also, a therapy may be applied to a patient having acute PTSD and routinely the effect of the therapy is monitored and recorded. The recorded data provides the basis for the assessment whether the therapy applied is beneficial to the patient or not. Based on said assessment the person skilled in the art, in this case likely a clinician, will be able to adjust the currently applied therapy, e.g., by increasing/decreasing dosage regimen or dosage amount of the therapy, or decide to completely switch to another therapy.

As is seen from a comparison of the chemical structure of resibufogenin and chemical structure of marinobufagenin, the difference between the two compounds is the absence of an hydroxyl group at the β-5 position in the resibufogenin structure.

The resibufogenin may be introduced into the patient by at least one method selected from the group consisting of intravenously, intraperitoneally, intramuscularly, intrathecally, subcutaneously, orally, intraoperatively, topically and during brain surgery by introducing it directly into brain tissue.

Assuming that the initial monitoring of the marinobufagenin indicated that a propensity for PTSD exists and treatment with resibufogenin or other MBG antagonist can be administered in order to ameliorate PTSD. This test may be repeated periodically and compared with at least one prior test until the marinobufagenin levels approach or reach the levels of normal patients. Additional doses of resibufogenin or other MBG antagonist may be administered during the course of treatment.

### II. Methods of Detecting Marinobufagenin (MBG)

MBG can be detected using a variety of assays including, but not limited to immuno-detection, microchip, or lateral flow based methods. Immuno-based assays include, but are not limited to radiolabeled, enzyme, fluorescence, dot blot, chemiluminescence, dip-stick, or biosensor assays. Methods for measuring MBG in the blood or urine can provide sensitivities in the pg/ml (picograms per milliliter) range or pg/mg creatinine, respectively. In certain aspects the MBG detection methods employ an MBG specific ELISA assay. In certain aspects the MBG assay discriminates between MBG and resibufogenin (RBG). In certain aspects, antibodies are used to detect the presence of MBG in an original or processed sample obtained from a subject (Abi-Ghanem et al., Journal of Immunoassay and Immunochemistry 2011, 32:31-46). Samples obtained from a subject may include, for example, cells, tissue, blood, serum, or urine. For example, a sample can be blood or urine collected from a subject. A sample can be analyzed directly or extracted before analysis.

1. A method of identifying a patient at risk of developting Post-Traumatic Stress Disorder (PTSD), comprising:
obtaining a sample from the patient;
measuring the level of marinobufagenin in the sample, wherein elevation of the level of marinobufagenin in the sample relative to a reference level is indicative of a propensity for PTSD in the patient.

2. The method of claim 1, wherein the reference level is the level of marinobufagenin in a sample from a subject that is known not to have PTSD.

3. The method of claim 2, wherein the reference level is a range of values of marinobufagenin in samples obtained from a population of subjects that do not have PTSD.

4. The method of claim 1, wherein elevation of the level of marinobufagenin is defined as an elevation of at least 30 percent compared to the reference level.

5. A method of treating a patient who has been diagnosed as having or is at risk of developing Post-Traumatic Stress Disorder (PTSD) comprising, administering to the patient a therapeutically effective dose of a marinobufagenin (MBG) antagonist.

6. The method of claim 5, wherein the MBG antagonist is resibufogenin (RBG).

7. The method of claim 5 including administering a second dose of the MBG antagonist.

8. The method of claim 5, wherein diagnosing PTSD comprises (i) obtaining a sample from the patient at risk of developing PTSD; and (ii) measuring the level of marinobufagenin in the sample, wherein elevation of the level of marinobufagenin in the sample relative to a reference level is indicative of a propensity for PTSD in the patient.

9. The method of claim 8, wherein the marinobufagenin level is at least about 30 percent above a reference level.

10. The method of claim 9, wherein the sample is a blood or urine sample.

11. The method of claim 10, wherein the blood sample is a serum or plasma sample.

12. The method of claim 8, wherein the sample is a urine sample.

13. The method of claim 8, wherein the sample is a cerebrospinal fluid sample.

14. The method of claim 8, further comprising periodically repeating the diagnostic method on the patient samples, and comparing the most current marinobufagenin concentration from the repeated test with at least one prior test of a patient sample.

15. The method of claim 5, wherein the MBG antagonist is administered by intravenous, intraperitoneal, intramuscular, intrathecal, subcutaneous, or oral administration.

16. A method of reducing the risk of PTSD in a patient who is suspected to have a propensity for developing PTSD, comprising administering to the patient a therapeutically effective amount of a MBG antagonist.
